(19) United States
(12) Patent Application Publication
(10) Pub. No.: US 2007/0015239 A1
(43) Pub. Date: Jan. 18, 2007

(54) Correlation between the fucose content/galactose content ratio of anti-rhesus-d and anti-hla-dr antibodies and the adcc activity

(75) Inventors: Nicolas Bihoreau, Orsay (FR); Christophe De Romeuf, Lille (FR); Sylvie Jorieux, Villeneuve D'Ascq (FR); Emmanuel Nony, Antony (FR); Dominique Bourel, La Madeleine (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Laboratoire Francais du Fractionnement et des Biotechnologies, Les Ulis (FR)

(21) Appl. No.: 10/575,333

(22) PCT Filed: Oct. 20, 2004
(86) PCT No.: PCT/FR04/02686

§ 371 Date: Apr. 12, 2006

(30) Foreign Application Priority Data
Oct. 20, 2003 (FR) 0312229

(51) Int. Cl. C12P 21/06; C07H 21/04; C12N 9/00; C07K 16/40; C12N 5/06; C12P 21/08

(52) U.S. Cl. 435/69.1; 435/328; 435/320.1; 435/188.5; 530/388.26; 536/23.5

(57) ABSTRACT

The present invention relates to monoclonal antibodies with high ADCC activity, characterised in having glycannic structures with a ratio (fucose content/galactose content) of 0.6 or less on the glycosylation site thereof in the Fc region. The invention also relates to pharmaceutical compositions, containing said monoclonal antibodies with high effector activity.

The present invention relates to compositions of monoclonal antibodies with high ADCC activity and for which the fucose content/galactose content ratio of the glycanic structures present on their glycosylation sites in the Fc region, is less than or equal to 0.6. The invention also relates to pharmaceutical compositions comprising said monoclonal antibodies having a high effector activity.

Very widespread passive immunotherapy is based on administration of antibodies, in particular immunoglobulins of the IgG type, directed against a cell or a given substance. Passive immunotherapy by means of monoclonal antibodies has given encouraging results. However, if the use of monoclonal antibodies has several advantages, like for example an assurance of the product's safety as to the absence of any infectious contamination, it may prove to be difficult to obtain an effective monoclonal antibody on the other hand.

Type G immunoglobulins (IgG) are heterodimers consisting of 2 heavy chains and 2 light chains, bound together by disulfide bridges. Each chain at the N-terminal position consists of a variable portion specific to the antigen against which the antibody is directed, and at the C-terminal position, consists of a constant portion inducing the effector properties of the antibody.

The association of the variable portions and of the CH₁ and CL domains of the heavy and light chains forms the Fab portions, which are connected to the Fc region (constant portion of the heavy chain) via a region with exceptional flexibility (a transition region) thereby allowing each Fab to be fixed to its antigen target whereas the Fc region remains accessible to effector molecules such as the FcγR receptors and the Clq.

The Fc region consists of 2 globular domains named CH₂ and CH₃. Both heavy chains closely interact at the CH₃ domains whereas at the CH₂ domains, the presence on each of both chains, of a biantennary N-glycane of the lactosaminic type, bound to Asn 297, contributes to a separation of both domains.

Many studies have shown that glycosylation of the Fc region is essential for the biological activity of IgGs, particularly for cellular lysis mediated by the complement (CDC) and cellular cytotoxicity depending on the antibody (ADCC). Thus, it was demonstrated that aglycosylated IgGs obtained by directed mutagenesis or by cultivating cells producing the antibody in the presence of tunicamycin, lose their capability of activating the complement and of fixing the FcγR receptors (Nose and Wigzell, 1983; Tao and Morrison, 1989).

More specific studies on the role of each monosaccharide have shown that attachment of a residue of N-acetylglucosamine (GlcNac) at a bisecting position leads to enhancing the ADCC activity of IgGs (Umana et al., 1999; Davies, 2001). On the other hand, the effect of whether galactose residues are present or not in the oligosaccharide bound to Asn297 is more controversial. If the presence of galactose residues was described as essential for the effector function of IgGs (Tsuchiya et al., 1989; Furukawa and Kobata, 1991: Kumpel et al., 1994), other authors have shown that the absence of galactose residues did not change the functional activity of IgGs (Boyd et al., 1995; Wright and Morrison, 1998).

In Patent Application WO 01/77181, we demonstrated that glycosylation of the Fc region is essential for the biological activity of IgGs, particularly for CDC and ADCC activity. We show that a biantennary N-glycane of the lactosaminic type characterized by short chains, slight sialylation, slight fucosylation, terminal mannose residues and/or non-intercalating terminal GlcNac residues, is the common denominator of glycanic structures imparting high ADCC activity to monoclonal antibodies. Subsequently, our discovery was corroborated by studies of Shields et al., (2002) and Shinkawa et al. (2003).

Within the scope of the present invention, we observed that therapeutic anti-D polyclonal antibodies (NATEAD, WinRho) have very high ADCC activity, taking into account their fucose content.

This observation implies that the low fucose content is not per se the only factor which influences the antibodies' capability of activating the FcγR receptors, and notably FcγRIII.

By studying the full glycoside profile of polyclonal antibodies, we discovered an inverse relationship between the [fucose content/galactose content] ratio and the effector activity of the antibodies.

Indeed, if the antibody is highly fucosylated, it needs to be highly galactosylated in order to have optimum effector activity. A contrario, if the antibody is slightly fucosylated, the present galactose content should be such that the fucose content/galactose content ratio is less than 0.6 but preferably less than 0.5 or even 0.4 in order to have optimum effector activity.

In the light of the experimental results, we have therefore set up a method for preparing antibodies having an optimized fucose content/galactose content ratio, with which antibodies having high effector activity may be obtained. In other words, we propose new monoclonal antibodies having a specific oligosaccharide structure, notably as regards fucose and galactose residues, imparting high effector activity. On the other hand, we also propose antibodies for which the glycanic structure does not provide any activation of cytotoxic activity as well as methods for obtaining them.

### DESCRIPTION

Thus, in a first aspect, the invention relates to a method for preparing a humanized or human chimeric monoclonal antibody, with high effector activity, characterized in that it comprises the following steps:
a) producing and purifying monoclonal antibodies obtained from different sources, notably from cells, plants or non-human animals, possibly either genetically altered or transformed,
b) measuring the fucose content and the galactose content of the glycanic structures borne by the glycosylation site of the Fc region of said antibodies,
c) selecting antibodies for which the fucose content/galactose content ratio is less than or equal to 0.6, preferably 0.5 or 0.4.

By “a monoclonal antibody” a composition is meant which comprises monoclonal antibodies having an identical primary structure, except for the small proportion of antibodies having mutations which have occurred naturally, identical specificity and post-translational modifications, notably modifications of glycosylation, which may vary from one molecule to another. For the purposes of the present invention, the expressions “monoclonal antibody” or “composition of a monoclonal antibody” are synonyms.

The monoclonal antibodies of the invention may be prepared by conventional methods, such as the production of hybridomas as described by Köhler and Milstein (1975), the immortalization of human B lymphocytes by Epstein-Barr's virus (EBV), or more recent ones, such as the phage display technology, the use of a combinatorial library of human or transgenic animal antibodies, notably from the mouse, Xenomouse®; monoclonal antibodies may also be prepared by molecular engineering, notably for chimerizing or humanizing antibodies. For the purposes of the invention, glycane analysis may be for example carried out with High-Performance Capillary Electrophoresis with Laser-Induced Fluorescence (HPCE-LIF), or by means of any other glycane analysis method known to one skilled in the art.

With the method according to the invention, a monoclonal antibody having high effector activity and more particularly high functional activity of the ADCC type, may be obtained. On this account, effector activity means biological activities able to be attributed to the Fc region of an antibody. Examples of these effector functions include, without being limited thereto, Antibody-Dependent Cell-mediated Cytotoxicity (ADCC) activity, Complement-Dependent Cytotoxicity (CDC) activity, phagocytosis activity, endocytosis activity or even induction of cytokine secretion.

A “high” effector activity means an effector activity at least 20 times, 50 times, 60 times, 70 times, 80 times, or 90 times and preferably up to 100 times, or preferentially 500 times higher than the effector activity of antibodies of same specificity but for which the fucose content/galactose content ratio is larger than 0.6.

Preferentially, the fucose content/galactose content ration is between the values of 0.6 and 0.3, preferentially between 0.5 and 0.35. Indeed, considering the experiments conducted within the scope of the invention, it appears that a limiting ratio exists, i.e., a fucose content/galactose content ratio, below which the functional, notably ADCC activity, does no longer increase linearly when the ratio decreases. Therefore, it is particularly advantageous to conduct the method according to the invention so as to be between these limits.

For example, if the fucose content is between 35% and 45%, the galactose content may be between 70 and 99%. If the fucose content is between 20% and 35%, the galactose content is between 55% and 70%, or even between 60% and 99%.

For the purposes of the invention, the value of the ratio less than or equal to 0.6 also means a value larger than 0.6 by a few hundredths of a unit, for example 4 to 5 hundredths.

In a particular aspect of the invention, the antibodies obtained by the method according to the invention are produced in genetically modified cells by introducing at least one vector allowing antibodies to be expressed, these cells being eukaryotic or prokaryotic cells, notably cells from mammals, insects, plants, bacteria or yeasts.

Advantageously, the obtained antibody is a human immunoglobulin of the IgG type.

More advantageously, these cells may be genetically modified by introducing at least one vector allowing the expression of at least one polypeptide having glycosyl transferase activity. Preferentially this glycosyl transferase activity is galactosyl transferase activity, and notably beta(1,4)-galactosyl transferase or beta(1,3)-galactosyl transferase activity.

For the purposes of the invention, a “polypeptide having galactosyl transferase activity” means any polypeptide capable of catalyzing the addition of a galactose residue from the UDP-galactose to the GlcNAc residue in the non-reducing position of an N-glycane.

For the purposes of the invention, a “vector allowing the expression of a polypeptide having beta(1,4)-galactosyl transferase activity” means any vector comprising a polynucleotide allowing the expression of a polypeptide capable of synthetizing the disaccharide pattern Galbeta(1,4)-GlcNac, this polynucleotide may stem from species such as humans, mice, hamsters, cows, sheep, goats, pigs, horses, rats, monkeys, rabbits, chickens, for example. Sequences such as for example NM 001497, AB 024434, NM 003780, BC 053006, XM 242992, NM 177512, this list not being exhaustive, are available in banks of nucleotide and/or protein sequences such as Genbank.

For the purposes of the invention, a “vector allowing the expression of a polypeptide having beta(1,3)-galactosyl transferase activity” means any vector comprising a polynucleotide allowing the expression of a polypeptide capable of synthetizing the disaccharide pattern Galbeta(1,3)-GlcNac, this polynucleotide may stem from species such as humans, mice, hamsters, cows, sheep, goats, pigs, horses, rats, monkeys, rabbits, chickens, for example. Notably, the sequences coding for a beta(1,3)-galactosyl transferase stemming from species such as humans, mice, hamsters, cows, sheep, goats, pigs, horses, rats, monkeys, rabbits, chickens, for example are particularly suitable. Such sequences are available on Genbank, such as for example NM020981, AB084170, AY043479, this list not being restrictive.

A “glycosylation site of the Fc region of the antibodies” generally means both Asn297 residues according to the numbering of Kabat (Kabat database, http://immuno.bme.nwu.edu), but the invention is also directed to antibodies for which the amino acid sequences have been changed.

In a particular embodiment of the invention, the cells further have an activity relating to the synthesis and/or the transport of GDP-fucose and/or the activity of an enzyme involved in adding fucose to the oligosaccharide of the glycosylation site of the antibodies, either reduced or deleted. Advantageously, the enzyme involved in the synthesis of GDP-fucose is GMD (GDP-D-mannose 4,6-dehydratase), Fx (GDP-keto-6-deoxymannose 3,5-epimerase, 4-reductase) or GFPP (GDP-beta-L-fucose pyrophosphorylase), this list not being exhaustive. Advantageously, the enzyme involved in adding fucose is a fucosyl transferase. The involved protein in transporting GDP-fucose may advantageously be the human GDP-fucose transporter 1.

In a particular embodiment of the invention, it is possible, if the fucose and galactose contents measured in step b) give a ratio larger than 0.6, to defucosylate and/or add galactose residues to the antibodies before step c) so that said ratio becomes less than 0.6 but preferably less than 0.5 and even less than 0.4 in order to increase the functional activity of the antibodies. This defucosylation may be carried out by adding a fucosidase into the medium containing the antibody, which may be the storage medium. Addition of galactose residues may be carried out with any suitable means including adding a galactosyl transferase in the medium containing the antibody or in a solution containing the antibody and a donor substrate such as UDP-galactose, for example.

Advantageously, the cells used for applying the method according to the invention, stem from animal or human cell lines, these lines being notably selected from rat myeloma lines, notably YB2/0 and IR983F, human myeloma lines such as Namalwa or any other cell of human origin such as PERC6, CHO lines, notably CHO-K, CHO-Lec10, CHO-Lec1, CHO Pro-5, CHO dhfr-, CHO Lec13, or other lines selected from Wil-2, Jurkat, Vero, Molt-4, COS-7, 293-HEK, BHK, K6H6, NSO, SP2/0-Ag 14 and P3X63Ag8.653.

Advantageously, the antibody is an anti-Rhesus D (anti-D), anti-CD, anti-tumors, anti-virus, anti-CD20 or an anti-HLA-DR, more particularly from the antibodies of the Table 0 hereafter:

TABLE 0

| Name and | | | |
|---|---|---|---|
| trade name of | | | |
| the antibody | Company | Target | Indication |
| Edrecolomab | Centocor | anti-Ep- | colorectal cancer |
| PANOREX | | CAM | |
| Rituximab | Idec | anti CD20 | B cell lymphoma |
| RITUXAN | Licensed to | | thrombocytopenia |
| | Genentech/ | | purpura |
| | Hoffman La Roche | | |
| Trastuzumab | Genentech | anti HER2 | ovarian cancer |
| HERCEPTIN | Licensed to Hoffman | | |
| | La Roche/Immunogen | | |
| Palivizumab | Medimmune | | RSV |
| SYNAGIS | Licensed to Abott | | |
| Alemtuzumab | BTG | anti CD52 | leukemia |
| CAMPATH | Licensed to Schering | | |
| Ibritumomab | IDEC | anti CD20 | NHL |
| Tiuxetan | Licensed to Schering | | |
| ZEVALIN | | | |
| Cetuximab | Merck/BMS/ | anti EGFR | cancers |
| IMC-C225 | Imclone | | |
| Bevacizumab | Genentech/ | anti VEGFR | cancers |
| AVASTIN | Hoffman La Roche | | |
| Epratuzumab | Immunmedics/ | anti CD22 | cancers: |
| | Amgen | | non-hodgkinian |
| | | | lymphoma |
| Hu M195Mab | Protein Design Labs | anti CD33 | cancers |
| MDX-210 | Immuno-Designed | ND | cancers |
| | Molecules | | |
| BEC2 | Imclone | anti GD3 | cancers |
| Mitumomab | | | |
| Oregovomab | Altarex | anti CA125 | ovarian cancer |
| OVAREX | | | |
| Ecromeximab | Kyowa-Hakko | anti GD3 | malign melanoma |
| KW-2971 | | | |
| ABX-EGF | Abgenix | EGF | cancers |
| MDX010 | Medarex | Anti CD4R | Cancers |
| XTL 002 | XTL | ND | antiviral: HCV |
| | Bio-pharmaceuticals | | |
| H11 SCFV | viventia biotech | ND | cancers |
| 4B5 | viventia biotech | anti GD2 | cancers |
| XTL 001 | XTL | ND | antiviral: HBV |
| | Bio-pharmaceuticals | | |
| MDX-070 | MEDAREX | Anti-PSMA | prostate cancer |
| TNX-901 | TANOX | anti IgE | allergies |
| IDEC-114 | IDEC | Protein C | non-Hodgkinian |
| | | inhibition | lymphoma |

This list is however not restrictive.

A second object of the invention is to provide a method for increasing effector activity, notably ADCC activity, of a composition of immunologically functional molecules, comprising increasing the galactose content and/or reducing the fucose content of the composition of molecules.

The term “immunologically functional molecules” is meant to designate molecules capable of reacting to any contact with any immunogen by demonstrating immunological capability. These molecules in the native condition may have good effector activity, for example ADCC or poor effector activity. They have a Fc region including a glycosylation site. For this purpose, these functionally immunologic molecules preferentially are antibodies, advantageously monoclonal or polyclonal antibodies.

The molecules in the native condition may have high fucose content. More particularly, in this case, it is advantageous to proceed with an increase of the galactose content of these molecules or antibodies.

In an embodiment of the invention, reduction of the fucose content is achieved by defucosylation of the molecules of the composition through the action of a fucosidase. This defucosylation may be carried out by a α1,6-fucosidase. Fucosidases extracted from bovine kidneys or from Charonia lampas have this specificity.

In another embodiment of the invention, the increase in the galactose content of the molecules of the composition is due to galactosylation of the composition by the action of a galactosyl transferase.

In a particular embodiment of the invention, enzymes for defucosylation and enzymes for galactosylation are both caused to act.

As an alternative to the enzymatic treatment, the composition of immunologically functional molecules may be purified by a series of chromatographies on lectins which enrich the composition with lowly-fucosylated antibodies and/or highly-galactosylated antibodies.

As an example, the solution comprising the composition of immunologically functional molecules which advantageously are antibodies, is passed over a lectin column (for example an LA-LCA or LA-AAL column, Shimadzu Corporation) connected to a HPLC system. The solution is separated into a non-absorbed fraction and an adsorbed fraction. A glycane analysis of the non-adsorbed and absorbed fractions is performed: the oligosaccharides, cleaved from the protein portion by enzymatic action, are marked with APTS and separated by HPCE-LIF and quantified. The areas of the peaks are calculated: antibodies having fucose-free glycanes may thereby be separated and selected. The selected fraction is then passed (which may be issued from the non-absorbed fraction or from the absorbed fraction) either on a hydrophobic column of the phenyl-5PW type (prepared by Tosoh Corporation) or on a second lectin column (LA-RCA 120 or LA-WGA, Seikagaku America). The fractions for which the fucose content/galactose content ratio is less or equal to 0.6 may thereby be selected accurately.

A third object of the invention is a cell, preferentially derived from the YB2/0 cell line, in which at least one vector coding for an antibody molecule is introduced, said cell producing a monoclonal antibody having a fucose content/galactose content ratio of oligosaccharides from the glycosylation site of the Fc region, less than or equal to 0.6. Preferentially this ratio is less than 0.5 or even 0.4. In a preferred aspect of the invention, this ratio is between 0.6 and 0.3.

In a preferred aspect of the invention, this cell is transfected with an expression vector coding for a galactosyl transferase, notably for a beta(1,4)-galactosyl transferase or a beta(1,3)-galactosyl transferase. Advantageously, this cell expresses or overexpresses a recombinant galactosyl transferase.

The YB2/0 line naturally expresses galactosyl transferases of the beta(1,4) and beta(1,3) family. Moreover, this cell line is known for producing antibodies having low fucose content (WO 01/77181, LFB). However, the cell according to the invention has the advantage of overexpressing galactosyl transferase, which has the effect of varying the fucose content/galactose content ratio of the antibodies produced by the modified cell relatively to the antibodies produced by the unmodified line. Therefore, as the antibody is naturally lowly fucosylated, an increase of its galactose content further lowers its fucose content/galactose content ratio, which has the effect of further optimizing its ADCC activity.

Advantageously, the galactosyl transferase is coded by a sequence originating from humans, mice, hamsters, cows, sheep, goats, pigs, horses, rats, monkeys, rabbits, or chickens, this list not being restrictive. More particularly, the coding sequence is the NM 001497, AB 024434, NM 003780, BC 053006, XM 242992 or NM 177512 sequence.

Thus, the invention also relates to a method for preparing monoclonal antibodies for which the glycanic structures borne by the glycosylation site of the Fc region have a fucose content/galactose content ratio less than or equal to 0.6, preferentially less than 0.5 or even 0.4, comprising growing the cell described earlier in a culture medium and under conditions allowing expression of said vectors.

Alternatively, antibody compositions such as those defined above, may be prepared by means of one or more chromatography steps by using any molecule capable of trapping with specificity the fucose, galactose or oligosacchlarides which comprise them. As such, separation over lectin may be used, as illustrated hereinbefore.

Also, the invention relates to therapeutic antibodies having high effector activity, capable of being obtained from the methods described earlier or even obtained from the described methods, these antibodies being characterized in that they have on their glycosylation site of the Fc region, glycanic structures having a fucose content/galactose content ratio less than 0.6, preferentially less than 0.5 or even 0.4.

More advantageously, these are therapeutic monoclonal antibodies capable of being obtained from the previous method, said antibodies having reinforced ADCC activity, as an example, monoclonal anti-Ds having an ADCC activity equal to or larger than that of polyclonal antibodies. This reinforced ADCC activity is at least equal but preferentially larger than that of the polyclonal or monoclonal (of same specificity) therapeutic antibody expressed in the CHO DG44 or DxB11 line.

Advantageously, these may be IgGs, for example chimeric, humanized or human IgG1s or IgG3s, or IgGs having a human Fc region. Preferentially, these antibodies are human IgGs or any chimeric molecule including a human Fc region.

In the same order of ideas, the invention relates to a pharmaceutical composition comprising an antibody as described earlier.

Also, the invention relates to a pharmaceutical composition comprising at least 50%, preferentially 60%, 70%, 80% or even 90% or 99% of a monoclonal or polyclonal antibody for which the glycanic structures borne by the glycosylation site of the Fc region have a fucose content/galactose content ratio less than 0.6, preferentially less than 0.5 or even 0.4. Preferentially, the ratio is between the values 0.6 and 0.3, and more particularly between 0.5 and 0.35.

The compositions according to the invention preferentially include an antibody directed against a non-ubiquitous normal antigen, notably a Rhesus factor, such as the Rhesus factor (D) of the human red blood cell, or an antigen of a pathological cell or of a pathogenic organism for humans, in particular against an antigen of a cancer cell. The antibodies are further preferentially IgGs.

Another object of the invention relates to the use of an antibody according to the invention for preparing a drug intended for treating allo-immunization, notably the hemolytic disease of the newborn child.

Another object of the invention relates to the use of an antibody according to the invention for preparing a drug intended for treating auto-immune diseases, cancers, and infections by pathogenic agents, notably for treating diseases eluding the immune response notably selected from Sezary's Syndrome, solid cancers, notably for which the antigenic targets are weakly expressed, notably breast cancer, pathologies related to the environment notably aimed at persons exposed to polychlorinated biphenyls, infectious diseases, notably tuberculosis, chronic fatigue syndrome (CFS), parasite infections such as for example schistosomulas, and viral infections.

Further, the antibody according to the invention may be used for preparing a drug intended for treating cancers of positive class II HLA cells such as melanomas, acute lymphoid leukemias of B and T cells, acute and chronic myeloid leukemias, Burkitt's lymphoma, Hodgkin's lymphoma, T-cell lymphomas and non-Hodgkinian lymphomas.

The antibodies of the invention may be selected from antibodies appearing in Table 0.

Advantageously, the antibody is an anti-HLA-DR or an anti-CD20.

In another aspect of the invention, the antibody according to the invention is used for manufacturing a drug intended to induce expression of at least one cytokine selected from IL-1α, IL-1β, IL-2, IL-3, IL-4; IL-5, Il-6, IL-12, IL-18, IL-21, TGFβ1, TGFβ2, TNFα, TNFβ, INFγ and IP10 by the natural effector cells of the immune system, said drug being notably useful for treating cancer and viral, bacterial or parasite infections.

In another particular aspect of the invention, the antibody according to the invention is used for manufacturing a drug intended for treating patients having one of the polymorphisms of CD16, in particular V/F158 or F/F158, notably patients in a condition of therapeutic failure with the presently available antibodies or subject to undesirable secondary effects.

In an additional aspect, the invention also relates to a method for preparing a chimeric, humanized or human monoclonal antibody, having low effector activity, notably low functional activity of the ADCC type, characterized in that it comprises the following steps:
a) producing and purifying monoclonal antibodies obtained from different sources, notably from cells, plants, or non-human animals, possibly either genetically modified or transformed,
b) measuring the fucose content and the galactose content of the glycanic structures borne by the glycosylation site of the Fc region of said antibodies,
c) selecting antibodies for which the fucose content/galactose content ratio is larger than 0.6, preferentially larger than 1.2.

As such, the definitions of the effector activity of a monoclonal antibody are the same as those given earlier.

Moreover, “low effector activity” means an effector activity at least 20 times, 50 times, 60 times, 70 times, 80 times or 90 times and preferably up to a 100 times, or preferentially 500 times less than the effector activity, notably less than the ADCC type functional activity of antibodies with the same specificity but for which the fucose content/galactose content ratio is less than 0.6.

In a complementary aspect, the invention is therefore directed to antibodies with low ADCC activity and to the compositions which comprise them, characterized in that their glycosylation site (Asn 297) of the Fc region has a fucose content/galactose content ratio larger than 1.2.

These antibodies are useful for preparing drugs for treating and/or preventing auto-immune diseases, notably immunologic thrombopenic purpura (PTI), allo-immunizations, graft rejections, allergies, asthma, dermatites, urticarias, erythemas, and inflammatory diseases.

In a particular aspect of the invention, the antibodies are produced in genetically modified cells by introducing at least one vector allowing expression of said antibodies, said cells being eukaryotic or prokaryotic cells, notably cells from mammals, insects, plants, bacteria or yeasts.

In an embodiment of the invention, the cells are genetically modified by introducing at least one vector allowing expression of at least one polypeptide having glycosyl transferase activity, preferentially fucosyl transferase activity, and notably α1,6-fucosyl transferase activity.

In another embodiment of the invention, the cells have an activity relating to the synthesis and/or the transport of UDP-galactose, and/or the activity of an enzyme involved in adding galactose to the oligosaccharide of the glycosylation site of the antibodies is reduced or deleted. Advantageously, this enzyme involved in adding galactose is a β1,4-galactosyl transferase.

Advantageously, the cells both have glycosyl transferase activity, preferentially glycosyl transferase activity, and an activity relating to the synthesis and/or the transport of UDP-galactose and/or the activity of an enzyme involved in adding galactose to the oligosaccharide of the glycosylation site of the antibodies, either reduced or deleted.

In an embodiment of the invention, it may be provided that if in step b), the measured ratio is less than 0.6, fucosylation is performed and/or the galactose residues are removed from said antibody before step c), so that the fucose content/galactose content ratio becomes larger than 0.6.

Advantageously, degalactosylation is carried out by adding a galactosidase in the medium containing the antibody.

Advantageously, addition of fucose residues is carried out by adding a fucosyl transferase into the medium containing the antibody.

More advantageously, the antibody is a human immunoglobulin of the IgG type. Advantageously, the antibody is directed against a CD, a marker for differentiating human blood cells or against a pathogenic agent or its toxin listed as being particularly dangerous in the case of bioterrorism, notably Bacillus anthracis, Clostridium botulium, Yersinia pestis, Variola major, Francisella tularensis, filoviruses, arenaviruses, Brucella species, Clostridium perfringens, Salmonella, E. coli, Shigella, Coxiella burnetii, ricin toxin, Rickettsia, viral encephalitis viruses, Vibrio cholerae or hantavirus.

Another object of the invention relates to a method for reducing the activity of a composition of immunologically functional molecules, comprising the increase in the fucose content and/or the reduction in the galactose content of said composition.

Advantageously, the immunologically functional molecules are monoclonal or polyclonal antibodies.

In a particular aspect, the increase in fucose content is due to fucosylation of said composition through the action of a fucosyl transferase, preferentially a α1,6-fucosyl transferase.

In another particular aspect, the reduction of the galactose content of said composition is due to degalactosylation of the composition through the action of a galactosidase, preferentially one or more β-galactosidases.

More advantageously, both fucosylation and degalactosylation of this composition are performed.

Thus, an object of the invention relates to a composition of antibodies capable of being obtained from the methods according to the invention described above, or to an antibody composition obtained from one of these methods.

An additional object of the invention is the use of this antibody composition for preparing a drug intended for treating and/or preventing autoimmune diseases, and notably PTI, allo-immunization, graft rejections, allergies, asthma, dermatites, urticarias, erythemas, or inflammatory diseases, this list not being exhaustive.

Finally, the invention relates to a method for controlling the activity of a composition of immunologically functional molecules, comprising the regulation of the fucose content/galactose content ratio of the oligosaccharides from the glycosylation site of the Fc region of the antibodies.

Other aspects and advantages of the invention will be described in the examples which follow showing the “fucose effect” modulation by galactose, which should be considered as illustrative and which do not limit the scope of the invention.

### DESCRIPTION OF THE FIGURES

FIG. 1: Glycanic structures present on the glycosylation site of the Fc region of different anti-Rh(D) antibodies.

This figure illustrates the percentages of different glycanic forms borne by the Asn297 residues of 3 anti-Rh(D) antibodies: anti-D IgG1 of WinRho (black histograms), monoclonal EMAB2 antibody (white histograms) and anti-D1 (hatched histograms).

FIG. 2: Correlation line between the fucose content/galactose content ratio and the ADCC activity of anti-Rh(D) antibodies.

FIG. 3: Effect of galactose content on the ADCC activity of anti-Rh(D) antibodies.

This figure illustrates the lysis percentage of Rh(D+) erythrocytes induced by degalactosylated (Degal.) or non-degalactosylated (control) anti-Rh(D) polyclonal antibodies in the presence of polyvalent IgGs (Tegeline, LFB) at the concentration of 0.5 and 2.5 mg/ml.

FIG. 4: CD16 activation of degalactosylated anti-Rh(D) monoclonal antibodies.

This figure illustrates the % of CD16 activation induced by the presence of degalactosylated (white histograms) or non-degalactosylated (control, black histograms), EMAB2 and HH01 anti-Rh(D) monoclonal antibodies.

FIG. 5: CD16 activation of galactosylated anti-Rh(D) monoclonal antibodies.

This figure illustrates CD16 activation induced by the EMAB2 and AMAB3 anti-Rh(D) monoclonal antibodies, before (control, black histograms) and after in vitro galactosylation by bovine β1,4-galactosyl transferase (white histograms).

FIG. 6: Clearance curves of radio-labelled erythrocytes, either sensitized or not by anti-Rh(D) antibodies.

This figure illustrates the tracking of radioactivity, expressed as a %, contained in the blood of volunteers who have been re-injected with a volume of Cr⁵¹ radio-labelled erythrocytes either unsensitized (♦, ⋄) or sensitized by the therapeutic preparation of Rhophylac™ polyclonal antibodies(●) or by the EMAB2 monoclonal antibody (▪, ▴, Δ). The EMAB2 antibody was tested in 3 volunteers (008, 009, and 010).

FIG. 7: Effect of degalactosylation of anti-HLA DR monoclonal antibodies expressed in the YB2/0 and CHO-DG44 cell lines on CD16 activation.

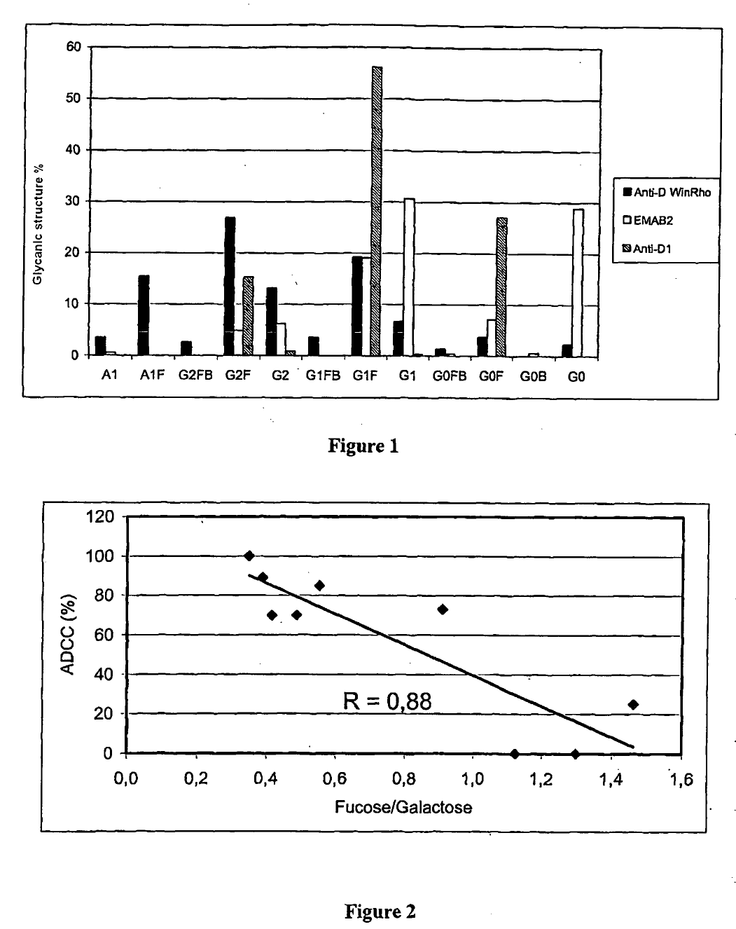

This figure illustrates the amount, expressed in pg/ml, of Il-2 secreted by Jurkat CD16 cells, the CD16 receptor of which has been activated, in the presence of Raji cells bearing on their membrane HLA DR molecules, by native (solid lines) or degalactosylated (dotted lines) anti-HLA DR chimeric antibodies.

### EXAMPLES

### Example 1

Correlation Between Fucose Content/Galactose Content Ratio and ADCC Activity of a Cohort of Anti-Rh(D) Antibodies.

We proceeded with measuring the fucose content, and then the galactose content of different monoclonal and polyclonal antibodies directed against the Rhesus (Rh) (D) antigen. From this, we inferred the ratio between both of them, and measured the ADCC activity relating to each antibody.

1. Production of Anti-Rh(D) Monoclonal Antibodies

Monoclonal antibodies stem from the transformation by EBV, of B lymphocytes from a negative Rh(D) human donor, immunized with erythrocytes bearing the Rh(D) antigen. Two clones were selected from this transformation:
1) one of the clones was merged with the K6H6-B5 human/mouse heteromyeloma; clone HH01 was selected from this fusion.
2) the RNAs coding for the anti-Rh(D) antibody were extracted from the other clone in order to prepare a vector for expressing the heavy chain and the light chain of the antibody.

This expression vector was used for transfecting the YB2/0 cell line giving rise to the EMAB1, EMAB2, EMAB3 and EMAB4 antibodies on the one hand and the following CHO lines on the other hand: DG44, K1 and Lec13 which synthetize the anti-D1, anti-D2 and anti-D3 antibodies, respectively.

2. Purification of Polyclonal Antibodies

The anti-Rh(D) polyclonal antibodies were immunopurified from a therapeutical product, WinRho (Cangene), by positive selection on Rh(D+) erythrocytes and then by negative selection on RhD(−) erythrocytes; finally an affinity chromatography step by using sepharose protein A gel allowed the recovered contaminants during the immunopurification on erythrocytes to be removed on the one hand and the IgG1s to be separated from the IgG3s on the other hand, as only IgG1s were used in the following tests.

3. Glycan Analysis by HPCE-LIF

The anti-Rh(D) monoclonal and polyclonal antibodies are desalted on a Sephadex G-25 (HiTrap Desalting, Amersham Biosciences) column, dried by evaporation and re-suspended in the buffer for hydrolyzing PNGase F (Glyko) in the presence of 50 mM of β-mercaptoethanol. After 16 hrs of incubation at 37° C., the protein portion is precipitated by adding absolute ethanol, and the supernatant which contains the N-glycanes, is dried by evaporation. The thereby obtained oligosaccharides are either directly marked with a fluorochrome, APTS (1-amino-pyrene-3,6,8-trisulfonate) or submitted to the action of specific exoglucosidases before marking them with APTS. Next, the marked oligosaccharides are injected on an N-CHO capillary and separated and quantified by capillary electrophoresis with detection of laser-induced fluorescence (HPCE-LIF).

Evaluation of the fucose content is performed by adding isolated fucosylated forms, or more specifically after simultaneous action of neuraminidase, β-galactosidase, and N-acetylexosaminidase, so as to obtain on the electrophoregram, 2 peaks corresponding to the pentasaccharide [GlcNac2-Man3] either fucosylated or not.

The fucose content expressed as a %, is calculated by using the following formula:

$$Fucose content = \frac{fucosylated [ GlcNac 2 - Man 3 ] \times 100}{[ GlcNac 2 - Man 3 + fucosylated GlcNac 2 - Man 3 ]}$$

The galactose content, expressed as a % is calculated by adding the percentages of the oligosaccharide forms containing galactose in the terminal position. The formula used is the following:
Galactose content=[(G1+G1B+G1F+G1FB)+2×(G2+G2F+G2B+G2FB)]

The fucose content/galactose content ratio is obtained by dividing the fucose content by the galactose content, the contents being calculated as described above.

4. Functional Activity of the Antibodies: ADCC

With the ADCC (Antibody-Dependent Cell-mediated Cytotoxicity) technique, it is possible to evaluate the antibody's capability of inducing lyses of Rh(D+) erythrocytes, in the presence of effector cells (mononucleated cells or lymphocytes).

Briefly, the erythrocytes of a red blood cell RhD(+) concentrate are treated with papain (1 mg/ml, 10 min at 37° C.) and then washed in 0.9% NaCl. The effector cells are isolated from a pool of at least 3 buffy-coats, by centrifugation on a Ficoll (Amersham), followed by an adherence step in the presence of 25% of SVF, so as to obtain a lymphocytes/monocytes ratio of the order of 9. In a microtitration plate (96 wells), one deposits per well: 100 μl of dilution of purified anti-Rh(D) antibodies (from 9.3 to 150 ng/ml), 25 μl of papained Rh(D+) erythrocytes (i.e. 1.10⁶), 25 μl of effector cells (i.e. 2.10⁶) and 50 μl of polyvalent IgGs (Tegeline, LFB) at usual concentrations of 2 and 10 mg/ml. The dilutions are made in 0.25% IMDM of fetal calf serum (SVF). After incubation for 1 night at 37° C., the plates are centrifuged, and then the released hemoglobin in the supernatant is measured via its peroxidase activity in the presence of a chromogenic substrate, 2,7-diaminofluorene (DAF). The results are expressed as a lysis percentage, 100% corresponding to total lysis of the erythrocytes in NH₄Cl (control 100%) and 0% to the reaction mixture without any antibodies (control 0%). The specific lysis is calculated as a percentage according to the following formula:
((OD sample−OD control 0%)×100)/(OD control 100%−OD control 0%)=ADCC %

A HPCE-LIF analysis of the oligosaccharides borne by the glycosylation site of the Fc region of anti-Rh(D) IgG1s was performed.

TABLE I

| | Fucose | Galactose | Fucose/ | |
|---|---|---|---|---|
| | content | content | galactose | ADCC |
| Antibody name | (%) | (%) | ratio | (%) |
| EMAB1 | 42.3 | 75.3 | 0.56 | 85 |
| EMAB2 | 25.6 | 72.9 | 0.35 | 100 |
| EMAB3 | 82.1 | 56.1 | 1.46 | 25 |
| EMAB4 | 40 | 60.6 | 0.66 | 73 |
| HH01 | 38.1 | 79.3 | 0.48 | 89 |
| Anti-D WinRho* | 76.1 | 120 | 0.63 | 70 |
| Anti-D1 | 100 | 88.8 | 1.13 | 0 |
| Anti (D2 | 95.7 | 71.8 | 1.33 | 0 |
| Anti-D3 | 24.3 | 58.4 | 0.42 | 70 |

*Immunopurified polyclonal anti-Ds

The values of the ratios [fucose content/galactose content] and the ADCC percentages, contained in Table I, are reported in abscissae and ordinates respectively in FIG. 2. The correlation coefficient of the plotted linear regression line is equal to 0.92.

Thus, there is a correlation between the [fucose content/galactose content] ratio and the ADCC activity of monoclonal and polyclonal anti-Rh(D) antibodies. The antibodies which have significant ADCC activity have a fucose content/galactose content ratio less than 0.6.

### Example 2

Comparison of the ADCC Activity of Anti-Rh(D) Polyclonal Antibodies Before and After Degalactosylation

1. Degalactosylation of Anti-Rh(D) Polyclonal Antibodies

The immunopurified polyclonal antibodies are dialyzed against the hydrolysis buffer (50 mM sodium acetate, pH 5.5 containing 4 mM of calcium chloride). The antibodies are desialylated and degalactosylated by incubation in the presence of 5 mU of neuraminidase (EC 3.2.1.18) from Vibrio cholerae (Calbiochem) and 9 mM of β-galactosidase (EC 3.2.1.23) produced by E. coli (Roche). The control, designated as “control”, consists of the same antibody preparation, treated as indicated above, but in the absence of neuraminidase and β-galactosidase. After 24 hrs of incubation at 37° C., the antibodies are stored at 4° C.

The antibodies generated in this example are separated into two fractions; one of the fractions is used for glycane analysis and the other fraction is reserved for measuring ADCC activity.

2. Glycane Analysis by HPCE-LIF

The procedure consists in desalting on a Sephadex-G25 column, the fraction of degalactosylated anti-Rh(D) polyclonal antibodies in order to remove the salts but also the free oses which may be present in the preparation. After denaturation and reduction of the antibodies, the glycanes are released through action of the endoglycosidase, PNGase F (Glyko). After 16 hrs of incubation at 37° C., the protein portion is precipitated by adding absolute ethanol and the supernatant which contains the N-glycanes, is dried by evaporation. In order to evaluate the contents of galactose and fucose contained in the thereby obtained oligosaccharides, the sample is submitted to the simultaneous action of sialidase and fucosidase or sialidase, β-galactosidase and N-acetylhexosaminidase, respectively, before marking with APTS. Next, the marked oligosaccharides are injected on an N-CHO capillary and separated and quantified by capillary electrophoresis with detection of laser-induced fluorescence (HPCE-LIF).

3. Measurement of ADCC Activity

Measurement of ADCC activity of the polyclonal antibodies before and after treatment with β-galactosidase was performed according to the method described in Example 1.

Thus, after action of the β-galactosidase, the glycanes of the Fc region of the anti-Rh(D) polyclonal antibodies have a residual galactose content of 17.7% and a fucose content equal to 68.5%. The fucose content/galactose content ratio of the degalactosylated polyclonal antibodies is therefore equal to 3.8.

The presence, in the ADCC test of polyvalent IgGs such as Tegeline in the present example, blocks the high affinity receptors (i.e. FcγRI or CD64), thereby making the lysis of Rh(D+) erythrocytes more specific to the interaction of anti-Rh(D) antibodies with FcγRIII receptors present on the effector cells.

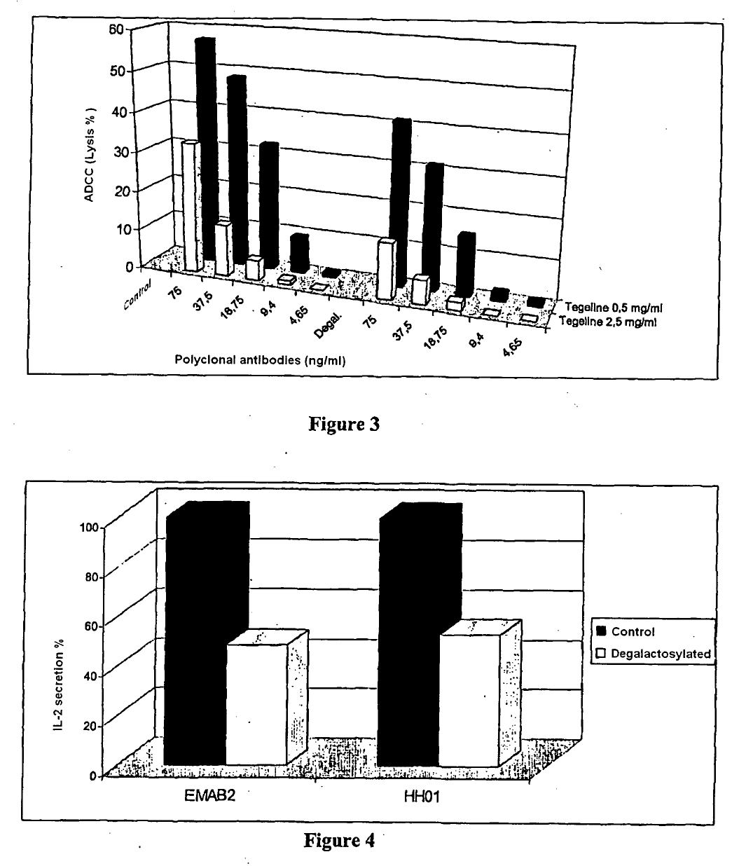

The results shown in FIG. 3 show that the ADCC activity of anti-Rh(D) polyclonal antibodies is dose-dependent on the one hand, and that increasing the amount of polyvalent IgGs in the reaction mixture causes a reduction in the lytic activity of the polyclonal antibodies. Further, the degalactosylated polyclonal antibodies have reduced ADCC activity relatively to that of the control antibodies.

TABLE II

| | |
|---|---|
| Degalactosylated | ADCC activity (%) |

| polyclonal | Tegeline | Tegeline |
|---|---|---|
| antibodies (ng/ml) | 0.5 mg/ml | 2.5 mg/ml |
| 75 | 72 | 42 |
| 37.5 | 65 | 46 |
| 18.75 | 47 | 40 |
| 9.4 | 23 | 0 |

The ADCC activity percentages of the degalactosylated anti-Rh(D) antibodies as compared with the control antibodies, i.e. having undergone the same incubation but in the absence of neuraminidase and β-galactosidase, are shown in Table II.

Thus, reduction in the ADCC activity of degalactosylated polyclonal antibodies as compared with the control antibodies is all the more significant since the amount of antibodies is small. Further, reduction in the activity of the degalactosylated polyclonal antibodies is more significant in the presence of a concentration of polyvalent IgGs of 2.5 mg/ml.

### Example 3

Measurement of the Activation of the CD16 Receptor Induced by Degalactosylated Anti-Rh(D) Monoclonal Antibodies

1. Degalactosylation of Anti-Rh(D) Monoclonal Antibodies

The antibodies are dialyzed against the hydrolysis buffer (50 mM sodium acetate, pH 5.5 containing 4 mM of calcium chloride). The antibodies are desialylated and degalactosylated by incubation in the presence of 5 mU of neuraminidase (EC 3.2.1.18) from Vibrio cholerae (Calbiochem) and 9 mU of β-galactosidase (EC 3.2.1.23) produced by E. coli (Roche). The control, designated as “control”, consists of the same antibody preparation, treated as indicated above, but in the absence of neuraminidase and β-galactosidase. After 24 hrs of incubation at 37° C., the antibodies are stored at 4° C.

The antibodies generated in this example are separated into two fractions; one of the fractions is used for glycane analysis and the other fraction is reserved for measuring the functional activity.

2. Measurement of CD16 Receptor Activation

The activation test for Jurkat CD16 cells measures the secretion of interleukine-2 (IL-2) induced by fixation of the Fc of antibodies on CD16 (FcγRIIIA) after binding the Fab to its antigen, present on the target cell. The IL-2 level secreted by Jurkat CD16 cells is proportional to the activation of the CD16 receptor.

50 μl of dilutions of antibodies, 50 μl of an erythrocyte suspension at 6.10⁵/ml, 50 μl of a suspension of Jurkat CD16 cells at 1.10⁶/ml and 50 μl of a PMA solution at 40 ng/ml are successively deposited in a 96-well microtitration plate. All the dilutions were carried out in an IMDM culture medium containing 5% SVF.

After 16 hrs of incubation at 37° C. and with 7% of CO₂, the microtitration plate is centrifuged and the amount of IL-2 contained in the supernatant is dosed with a commercial kit (Duoset, R&D). The secreted IL-2 levels are expressed in pg/ml.

The results are expressed as a CD16 activation percentage, the secreted IL-2 level in the presence of the control monoclonal antibody is considered to be equal to 100%.

The results of the glycane analysis performed by HPCE-LIF as described in Example 2, are gathered in Table III.

TABLE III

| Antibodies/ | EMAB2 | | HH01 | |
|---|---|---|---|---|
| Glycan | Control | Degalactosylated | Control | Degalactosylated |
| Fùcose(%) | 25.6 | 26.8 | 38.1 | 41.9 |
| Galactose(%) | 72.9 | 0 | 79.3 | 17.3 |
| Fuc/Gal ratio | 0.35 | N.A. | 0.48 | 2.42 |

It thus appears that the EMAB2 monoclonal antibody is totally degalactosylated when the HH01 antibody still contains 17.3% of monogalactosylated forms. After action of β-galactosidase, the fucose content/galactose content ratio of the EMAB2 and HH01 antibodies therefore becomes much larger than 0.6.

Degalactosylated anti-Rh(D) monoclonal antibodies have very reduced CD16 activation as compared with control antibodies (FIG. 4). Thus, the EMAB2 and HH01 monoclonal antibodies exhibit reduction of their capability of inducing CD16 activation by 52 and 47% respectively.

### Example 4

Measurement of CD16 Activation Induced by Galactosylated Anti-Rh(D) Monoclonal Antibodies

1. Galactosylation of the Antibodies

The antibodies are dialyzed against a 50 mM HEPES buffer, pH 7.20. The reaction mixture consists of the monoclonal antibody solution to which are added 10 mM of MnCl₂, 20 mM of UDP-galactose and 40 mU of bovine β1,4-galactosyl transferase (Calbiochem). After incubation at 37° C. for 24 hrs, the tubes are kept at 4° C. before use.

The control consists of the same antibody incubated under the same conditions except for the absence of UDP-Gal in the reaction medium.

The antibodies generated in this example are separated into two fractions; one of the fractions is used for glycane analysis and the other fraction is reserved for measuring ADCC activity.

2. Galactose Dosage by Lectin ELISA

Because of their recognition specificity, lectins were used in many applications of biology and medicine and notably in the analysis of glycanes by the ELISA technique. Lectin RCA1, which recognizes galactose bound in β1,4 was used for dosing the galactose present in the N-glycanes of the antibodies.

The monoclonal antibodies are immobilized in the wells of a microtitration plate. After heating for 20 min at 100° C. to denaturate the IgG molecules in order to make the N-glycanes of the Fc region accessible, the wells are incubated for 2 hrs at room temperature and under mild stirring in the presence of a biotinylated RCA₁ solution (Vector). After washing for removing the non-reacted lectin, the streptavidine peroxidase is added in each well, incubated for 1 hr, and the fixed lectin is measured at 492 nm after adding O-phenylene diamine. In parallel, the amount of fixed antibody in the wells of the microtitration plate is measured by a human anti-IgG antibody marked with peroxidase.

Next, the amount of fixed lectin is corrected by the amount of fixed antibody in the microtitration wells.

3. Measurement of CD16 Receptor Activation

The operating conditions used for measuring the activation of the CD16 receptor of the galactosylated monoclonal antibodies are identical with those described above.

The monoclonal antibodies described in the present example are anti-Rh(D) antibodies with the same primary sequence and produced by the YB2/0 cell. They differ by their functional activity, in connection with their α1,6-fucosylation rate which is 25% for EMAB2 and 53% for EMAB3.

After in vitro action of β-1,4-galactosyl transferase, the CD16 activation induced by the EMAB2 and EMAB3 monoclonal antibodies is increased by 10 and 54%, respectively (FIG. 5). Thus, the increase in galactosylation of the EMAB2 antibody which originally had very good effector activity, only induces a slight enhancement of the CD16 activation whereas the increase in galactosylation of the EMAB3 antibody, which is highly fucosylated, is expressed by a very significant enhancement of CD16 activity.

### Example 5

Study of the Clearance of Erythrocytes Sensitized by the EMAB2 Anti-Rh(D) Monoclonal Antibody

The EMAB2 anti-Rh(D) monoclonal antibody was evaluated in a clinical phase I test in order to compare clearance of erythrocytes sensitized by this antibody with that of erythrocytes sensitized by Rhophylac™, a therapeutic preparation of anti-Rh(D) polyclonal antibodies, used in clinics.

The erythrocytes of healthy volunteers are marked ex-vivo with chrome 51 (⁵¹Cr) and sensitized, i.e. incubated, in the presence of anti-Rh(D) antibodies, EMAB2 or Rhophylac™,in order to obtain a saturation level of 25% of the antigenic sites, before being re-injected into the volunteers.

Disappearance in the blood stream of the erythrocytes marked with ⁵¹Cr was followed by measuring radioactivity with a gamma counter on blood samples taken at 3, 15, 30 min and 1, 2, 4, 6, 8, 10, 24, 48, 72, 96 hrs after transfusion of the marked and sensitized erythrocytes. The blood sample taken at 3 min after transfusion of the erythrocytes represents 100% survival of the red corpuscles.

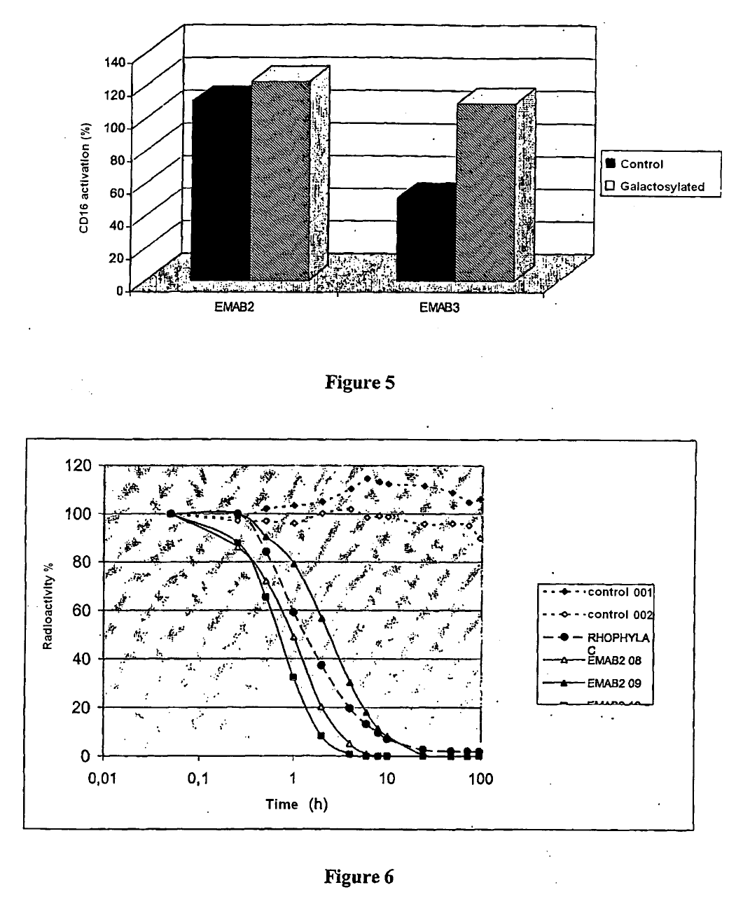

The results shown in FIG. 6 show that in the absence of sensitization of the radio-labelled erythrocytes by an antibody, the decrease of radioactivity measured over a period of time longer than 100 hrs, is less than 20%. However, when the erythrocytes are sensitized by a therapeutic preparation of polyclonal antibodies or by the EMAB2 monoclonal antibody, blood radioactivity decreases rapidly; ten hrs after the injection, there remains less than 10% of the injected radioactivity. Thus, the disappearance curve of erythrocytes sensitized by the EMAB2 monoclonal antibody has a profile similar to that of erythrocytes sensitized by the therapeutic preparation of Rhophylac™ polyclonal antibodies.

The EMAB2 monoclonal antibody for which the fucose content/galactose content ratio is equal to 0.4, has an activity in vivo, with regards to clearance of the pre-sensitized Rh(D+) erythrocytes, at least comparable to that of a therapeutic preparation of polyclonal antibodies.

Clinical studies performed under the same conditions but with another monoclonal antibody, called MonoD, gave very different results; at 25% saturation of the membrane antigenic sites, clearance induced by MonoD was only partial. Glycanic analysis of the MonoD antibody reveals the presence of a fucose content of 80% and a galactose content of 86%, i.e. a ratio equal to 0.93.

The comparison of these clinical results therefore shows that the anti-D monoclonal antibodies having a fucose content/galactose content ratio less than or equal to 0.6., have higher effectiveness on the clearance of erythrocytes, than that of antibodies for which the ratio is close to 1.

### Example 6

Modification of the Galactose Content of an Anti-HLA DR Monoclonal Antibody Expressed by CHO and YB2/0 Cell Lines

1. Producing the Anti-HLA DR Monoclonal Antibody

1.1. Construction of the Expression Vectors

The anti-HLA DR antibody used in these study stems from chimerization of the IgG2a isotype mouse antibody, expressed by the Lym-1 hybridoma (ATCC Hb-8612).

The RNA extracted from the hybridoma producing the mouse antibody was converted into cDNA. The mouse VK region was amplified by means of the K-Lym-Not1 and K-Lym-Dra3 primers and then cloned in the chimerization vector CK-Hu, digested beforehand by Not1 and Dra3, which contains the CK sequence of a human anti-D antibody and the DHFR selection gene.

The mouse VH region was amplified by means of primers H-Lym-Not 1 and H-Lym-Apa 1, and then cloned in the chimerization vector G1-Hu, digested beforehand by Not 1 and Apa 1, which contains the sequence G1 of a human anti-D antibody and the selection gene NEO.

The hEF-1a promoter and the 5′ UTR region of the hEF-1a gene containing the non-coding exon 1 and the first intron, was isolated from the commercial plasmid pEF/Bsd (Invitrogen) by Nhe 1 and Acc 65 I double digestion. In parallel, the RSV promoter present in the expression vectors described above, was deleted by Bgl II and Spe I double digestion and then replaced with the fragment Nhe I-Acc65 I.

1.2. Obtaining Stable Production Lines

The expression vectors pEF-Lym-dhfr-K-10 and pEF-Lym-neo-H-12 coding for the light chain and the heavy chain of the anti-HLA DR chimeric antibody, respectively, were used for co-transfecting, by electroportation, the CHO-DXB11 (ATCC No. CRL-11397) and YB2/0 (ATCC No.CRL-1662) lines.

After transfection, the cultivated cells are submitted to double selection pressure comprising deletion into nucleosides of the culture medium on the one hand and addition of G418 on the other hand. The resistant transformants to this double selection pressure were then cloned by limiting dilution.

The two selected clones are YB2/0-DR-4B7 for the YB2/0 expression cell line and DXB11-DR-22A10 for the CHO-DXB11 expression cell line.

1.3. Production and Purification of the Anti-HLA DR Chimeric Antibody

The YB2/0-DR-4B7 clone was grown in a cell-culture bioreactor of 10 litres (Biolafitte) in EM-SF1.1 medium, an EMS basic medium supplemented with insulin (1 μg/ml), iron citrate (50 μg/ml), HEPES (4 mg/ml) and Pluronic F68 (0.5 mg/ml).

The clone DXB11-DR-22A10 was grown in a cell-culture bioreactor of 10 litres (Biolafitte) in a CHO SFM4 utility medium (Perbio) supplemented with 2% hypoxanthine.

When cell viability is less than 50%, the culture media are collected, centrifuged, in order to remove the cells and the chimeric antibodies contained in the supernatant are purified by affinity chromatography on sepharose-protein A.

2. Degalactosylation

The anti-HLA DR chimeric antibodies were dialyzed against a 50 mM sodium acetate buffer, pH 5.50, containing 4 mM CaCl₂. The antibodies are degalactosylated by incubation in the presence of 5 mU of neuraminidase (EC 3.2.1.18) from Vibrio cholerae (Calbiochem) and 9 mU of β-galactosidase (EC 3.2.1.23) produced by E. coli (Roche). The control consists of the same antibody treated as indicated above but in the absence of neuraminidase and β-galactosidase. After 24 hrs of incubation at 37° C., the antibodies are stored at 4° C.

The antibodies generated in this example are separated into two fractions; one of the fractions is used for glycane analysis and the other fraction is reserved for measuring the functional activity.

3. Measurement of CD16 Activation

The Raji cell line is used as a target as it bears at its surface, the antigenic determinant of the HLA-DR histocompatibility major complex.

50 μl of antibody dilutions, 50 μl of a suspension of Raji cells at 6.10⁵/ml, 50 μl of a suspension of Jurkat CD16 cells at 1.10⁶/ml and 50 μl of a 40 ng/ml PMA solution were successively deposited in a 96-well microtitration plate. All the dilutions were made in the EMS culture medium containing 5% SVF.

After 16 hrs of incubation at 37° C. and with 7% of CO₂, the microtitration plate is centrifuged and the amount of IL-2 contained in the supernatant is dosed with a commercial kit (Duoset, R&D). The secreted IL-2 levels are expressed in pg/ml.

The results are expressed as a % of CD16 activation, the secreted IL-2 level in the presence of the control monoclonal antibody is considered to be equal to 100%.

The anti-HLA DR chimeric antibodies have very different glycanic structures as to whether they are expressed by the YB2/0 line or the CH0 DXB11 line. Thus, the fucose content/galactose content ratio for the antibody expressed by YB2/0 is equal to 0.37 whereas the ratio for the antibody expressed in CHO is much increased, since it is equal to 1.3.

CD16 activation of the native antibodies is consistent with the values of the fucose content/galactose content ratios; thus, IL-2 secretion induced by the anti-HLA DR antibody synthetized by YB2/0 and which has a ratio of 0.37 is twice that induced by the same antibody synthetized by CHO DXB11 but for which the ratio is equal to 1.3.

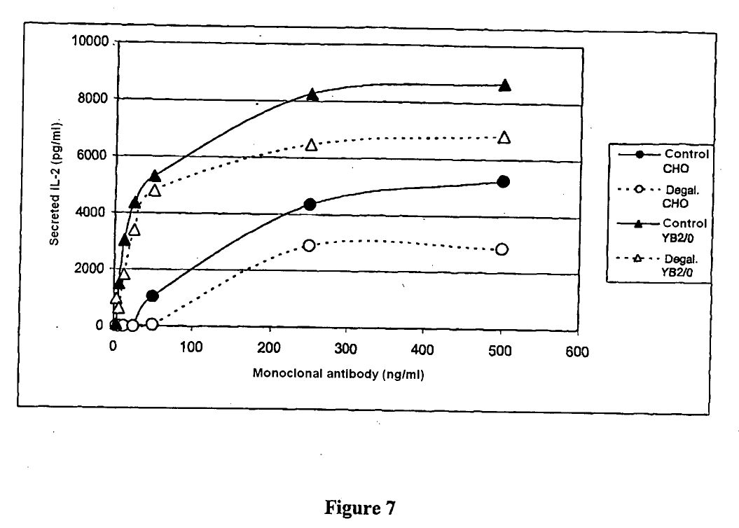

After action of β-galactosidase, the galactose content remaining on the N-glycane of the Fc region was determined by HPCE-LIF. Degalactosylation is nearly complete, the G1 form levels for the antibody produced by CHO and the G1B form level for the antibody produced by YB2/0 being 7% and 4.4%, respectively. This lowering of the galactose content is expressed by a significant reduction of CD16 activation as compared with the control antibodies, as shown in FIG. 7.

### REFERENCES

Boyd P N, Lines A C, Patel A K. The effect of the removal of sialic acid, galactose and total carbohydrate on the functional activity of Campath-1H. (1995) Mol. Immunol. 32, 1311-1318.
Davies J, Jiang L, Pan L Y, Labarre M J, Anderson D, Reff M. Expression of GnTIII in a recombinant anti-CD20 CHO production cell line: Expression of antibodies with altered glycoforms leads to an increase in ADCC through higher affinity, for FC gamma RIII. (2001) Biotechnol. Bioeng. 74, 288-294.
Furukawa K, Kobata A. IgG galactosylation—its biological significance ant pathology. (1991) Mol. Immunol. 28, 1333-1340.
Köhler G, Milstein C. Continuous cultures of fused cells secreting antibody of predefined specificity. (1975) Nature 256, 495-7.
Kumpel B M, Rademacher T W, Rook G A, Williams P J, Wilson I B. Galactosylation of human IgG monoclonal anti-D produced by EBV-transformed B-lymphoblastoid cell lines is dependent on culture method and affects Fc receptor-mediated functional activity.(1994) Hum. Antibodies Hybridomas 5, 143-451.
Nose M, Wigzell H. Biological significance of carbohydrate chains on monoclonal antibodies. (1983) Proc. Natl. Acad. Sci. USA 80, 6632-6636.
Shields R L, Lai J, Keck R, O'Connell L Y, Hong K, Meng Y G, Weiler S H A, Presta L G. Lack of fucose on human IgGl N-linked oligosaccharide improves binding to human FcγRIII and antibody-dependent cellular toxicity. (2002) J. Biol. Chem. 277, 26733-26740.
Shinkawa T, Nakamura K, Yamane N, Shoji-Hosaka E, Kanda Y, Sakurada M, Uchida K, Anazawa H, Satoh M, Yamasaki M, Hanai N, Shitara K. Absence of fucose but not presence of galactose or bisecting N-acetylglucosamine of human IgG1 complex-type oligosaccharides shows the critical role of enhancing antibody-dependent cellular cytotoxicity. (2003) J Biol Chem. 278, 3466-3473.
Tao M H, Morrison S L. Studies of aglycosylated chimeric mouse-human IgG. Role of carbohydrate in the structure and effector functions mediated by the human IgG constant region. (1989) J. Immunol. 143, 2595-2601.
Tsuchyia N, Endo T, Matsuta K, Yoshinoya S, Aikawa T, Kosuge E, Takeuchi F, Miyamoto T, Kobata A. Effects of galactose depletion from oligosaccharide chains on immunological activities of human IgG. (1989) J. Rheumatol. 16, 285-290.
Umana P, Jean-Mairet J, Moudry R, Amstutz H, Bailey J E. Engineered glycoforms of an antineuroblastoma IgG1 with optimized antibody-dependent cellular cytotoxic activity. (1999) Nat. Biotechnol. 17, 176-180.
Wright A, Morrison S L. Effect of C2-associated carbohydrate structure on Ig effector function Studies with chimeric mouse-human IgG1 antibodies in glycosylation mutants of chinese hamster ovary cells 1. (1998) J.Immunol. 160, 3393-3402.

1. A method for preparing a humanized or human chimeric monoclonal antibody, with high effector activity, comprising:
a) producing and purifying monoclonal antibodies obtained from different sources selected from the group consisting of cells, plants and non-human animals,
b) measuring the fucose content and the galactose content of the glycanic structures borne by the glycosylation site of the Fc region of said antibodies, and
c) selecting antibodies for which the fucose content/galactose content ratio is less than or equal 0.6.

2. The method of claim 1, wherein said antibodies are produced in genetically modified cells by introducing at least one vector allowing the expression of said antibodies, said cells being eukaryotic or prokaryotic cells selected from the group consisting of cells from mammals, insects, plants, bacteria and yeasts.

3. The method of claim 1, wherein said cells are genetically modified by introducing at least one vector allowing the expression of at least one polypeptide having a glycosyl transferase activity.

4. The method of claim 3, wherein said glycosyl transferase activity is a galactosyl transferase activity.

5. The method of claim 4, wherein said galactosyl transferase activity is a beta(1,4)-galactosyl transferase activity or a beta(1,3)-galactosyl transferase activity.

6. The method of claim 1, wherein said cells have an activity relating to the synthesis and/or the transport of GDP-fucose and/or to the activity of an enzyme involved in adding fucose to the oligosaccharide of the glycosylation site of the antibodies, either reduced or deleted.

7. The method of claim 6, wherein the enzyme involved in the synthesis of GDP-fucose is selected from the group consisting of GMD (GDP-D-mannose 4,6-dehydratase), Fx (GDP-keto-6-deoximannose 3,5-epimerase, 4-reductase) and GFPP (GDP-beta-L-fucose pyrophosphorylase).

8. The method of claim 6, wherein said enzyme involved in adding fucose is a fucosyl transferase.

9. The method of claim 1, wherein, if in step b), the measured ratio is larger than 0.6, a defucosylation is performed and/or galactose residues are added to said antibody before step c).

10. The method of claim 9, wherein said defucosylation is performed by adding a fucosidase in the medium containing the antibody.

11. The method according to of claim 1, wherein the addition of galactose residues is performed by adding a galactosyl transferase in the medium containing the antibody.

12. The method according of claim 1, wherein said cells stem from animal or human cell lines selected from the group consisting of rat myeloma YB2/0, rat myeloma IR983F, human myeloma Namalwa, cell of human origin, PERC6, CHO lines, CHO-K, CHO-Lec10 , CHO-Lec1, CHO Pro-5, CHO dhfr-, CHO Lec13 lines, Wil-2, Jurkat, Vero, Molt-4, COS-7, 293-HEK, BHK, K6H6, NSO, SP2/0-Ag 14 and P3X63Ag8.653.

13. The method of claim 1, wherein said antibody is an IgG type human immunoglobulin.

14. The method of claim 1, wherein said antibody is selected from the group consisting of an anti-Rhesus factor (anti-D), anti-CD, anti-tumors, anti-virus, anti-CD20 and anti-HLA-DR.

15. The method according of claim 1, wherein said effector activity is a ADCC type functional activity.

16. A method for increasing the effector activity of a composition of immunologically functional molecules, comprising the increase in galactose content and/or reduction in fucose content of the composition of molecules.

17. The method of claim 16, wherein said immunological functional molecules are monoclonal or polyclonal antibodies.

18. The method of claim 16, wherein said molecules have high fucose content in the native condition.

19. The method of claim 16, wherein the reduction in fucose content is due to a defucosylation of said composition through the action of a fucosidase.

20. The method of claim 16, wherein the increase in galactose content of said composition is due to a galactosylation of the composition through the action of a galactosyl transferase.

21. A cell derived from the YB2/0 cell line, in which at least one vector coding for an antibody molecule is introduced, said cell producing an antibody for which the fucose content/galactose content ratio of the oligosaccharides of the glycosylation site of the Fc region of the antibodies is less than or equal to 0.6.

22. The cell of claim 21, wherein said cell is transfected with an expression vector coding for a galactosyl transferase.

23. The cell of claim 21, wherein said galactosyl transferase is a beta(1,4)-galactosyl transferase or a beta(1,3)-galactosyl transferase.

24. The cell of claim 21, wherein said cell overexpresses said galactosyl transferase.

25. The cell of claim 21, wherein said galactosyl transferase is coded by a sequence originating from humans, mice, hamsters, cows, sheep, goats, pigs, horses, rats, monkeys, rabbits or chickens.

26. The cell of claim 25, wherein said sequence is the NM 001497, AB 024434, NM 003780, BC 053006, XM 242992, or NM 177512 sequence.

27. A method for preparing antibodies for which the glycanic structures borne by the glycosylation site of the Fc region has a fucose content/galactose content ratio less than or equal to 0.6, comprising the culture of a cell of claim 21 in a culture medium and under conditions allowing expression of said vectors.

28. Therapeutic antibodies having high effector activity obtained from the method of claim 1, wherein, said antibodies have on their glycosylation site of the Fc region, glycanic structures having a fucose content/galactose content ratio less than 0.6.

29. A pharmaceutical composition comprising an antibody according to claim 28 and at least one excipient.

30. A pharmaceutical composition comprising at least 50% of a monoclonal antibody for which the glycanic structures borne by the glycosylation site of the Fc region have a fucose content/galactose content ratio less than 0.6.

31. The pharmaceutical composition of claim 29, wherein the antibody is directed against a non-ubiquitous normal antigen, or an antigen of a pathological cell or on a pathogenic organism for humans.

32. The pharmaceutical composition of claim 29, wherein said antibodies are IgGs.

33. A method for treating allo-immunization, comprising administering the antibody of claim 28 to a patient in need thereof.

34. A method for treating an auto-immune disease, a cancer or an infection by a pathogenic agent comprising administering the antibody of claim 28 to a patient in need thereof.

35. A method for treating cancers of positive class II HLA cells, acute lymphoid leukemias of B- and T-cells, acute and chronic myeloid leukemias, Burkitt's lymphoma, Hodgkin's lymphoma, myeloid leukemias, T-cell lymphomas, and non-Hodgkinian lymphomas, comprising administering the antibody of claim 28 to a patient in need thereof.

36. The method of claim 33, wherein said antibody is an anti-HLA-DR or an anti-CD20.

37. A method for inducing expression of IL-1α, IL-1β, Il-2, IL-3, IL-4, IL-5, IL-6, IL-12, IL-18, IL-21, TGFβ1, TGFβ2, TNFα, TNFβ, IFBγ, or IP10 by natural effector cells of the immune system comprising administering the antibody of claim 28 to a patient in need thereof.

38. A method for for treating patients having one of the polymorphisms of CD16, in particular V/F158 or F/F158, notably patients in a condition of therapeutic failure with the presently available antibodies or subject to undesirable secondary effects comprising administering the antibody of claim 28 to a patient in need thereof.

39. A method for preparing a human or humanized chimeric monoclonal antibody having low effector activity, comprising:
a) producing and purifying monoclonal antibodies obtained from different sources selected from the group consisting of cells, plants, and non-human animals,
b) measuring the fucose content and the galactose content of the glycanic structures borne by the glycosylation site of the Fc region of said antibodies,
c) selecting antibodies for which the fucose content/galactose content ratio is larger than 0.6.

40. The method of claim 39, wherein said antibodies are produced in genetically modified cells by introducing at least one vector allowing expression of said antibodies, said cells being eukaryotic or prokaryotic cells selected from the group consisting of cells from mammals, insects, plants, bacteria, or yeasts.

41. The method according of claim 39, wherein said cells are genetically modified by introducing at least one vector allowing expression of at least one polypeptide having a glycosyl transferase activity.

42. The method of claim 41 wherein said glycosyl transferase activity is a fucosyl transferase activity.

43. The method according of claim 39, wherein said cells have an activity relating to the synthesis and/or the transport of UDP-galactose and/or to the activity of an enzyme involved in adding galactose to the oligosaccharide of the glycosylation site of the antibodies, either reduced or deleted.

44. The method of claim 43, wherein said enzyme involved in the addition of galactose is a galactosyl transferase.

45. The method of claim 39, wherein, if in step b), the measured ratio is less than 0.6, fucosylation is performed, and/or galactose residues are removed from said antibody before step c).

46. The method of claim 45, wherein said degalactosylation is performed by adding a galactosidase in the medium containing the antibody.

47. The method of claim 45, wherein addition of fucose residues is performed by adding a fucosyl transferase in the medium containing the antibody.

48. The method of claim 39, wherein said antibody is an IgG type human immunoglobulin.

49. The method of claim 39, wherein said antibody is directed against a CD, a differentiation marker of human blood cells or against a pathogenic agent or its toxins, listed as being particularly dangerous in the case of bioterrorism, selected from the group consisting of Bacillus anthracis, Clostridium botulium, Yersinia pestis, Variola major, Francisella tularensis, filoviruses, arenaviruses, Brucella species, Clostridium perfringens, Salmonella, E. coli, Shigella, Coxiella burnetti, ricin toxin, Rickettsia, viral encephalitis viruses, Vibrio cholerae and hantavirus.

50. The method of claim 39, wherein said effector activity is an ADCC type functional activity.

51. A method for reducing the activity of a composition of immunologically functional molecules, comprising the increase in the fucose content and/or the reduction in the galactose content of said composition.

52. The method of claim 51, wherein said immunologically functional molecules are monoclonal or polyclonal antibodies.

53. The method of claim 51, wherein the increase in the fucose content is due to fucosylation of said composition through the action of a fucosyl transferase.

54. The method according of claim 51, wherein the reduction in the galactose content of said composition is due to degalactosylation of the composition through the action of a galactosidase.

55. An antibody obtained from a method of claim 39.

56. A method for treating and/or preventing auto-immune diseases, allo-immunizations, notably PTI, graft rejection, allergies, asthma, dermatites, urticarias, erythemas, or inflammatory diseases comprising administering the antibody of claim 55 to a patient in need thereof.

57. A method for controlling the activity of a composition of immunologically functional molecules, comprising the regulation of the fucose content/galactose content ratio of the oligosaccharides of the glycosylation site of the Fc region of the antibodies.
